(19) United States
(12) Patent Application Publication
(10) Pub. No.: US 2025/0049369 A1
(43) Pub. Date: Feb. 13, 2025

(54) ELECTROMYOGRAPHY SENSOR

(71) Applicant: Life and Limb Pvt Ltd, Kanpur, Uttar Pradesh (IN)

(72) Inventors: Nishant AGARWAL, Ghaziabad (IN); Prachi KHARB, Chandigarh (IN)

(73) Assignee: Life and Limb Pvt Ltd, Kanpur, Uttar Pradesh (IN)

(21) Appl. No.: 18/717,974

(22) PCT Filed: Dec. 8, 2022
(86) PCT No.: PCT/IN2022/051060

§ 371 Date: Jun. 7, 2024

(30) Foreign Application Priority Data
Dec. 8, 2021 (IN) 202111057165

(51) Int. Cl. A61B 5/296; A61B 5/00

(52) U.S. Cl. CPC A61B 5/296; A61B 5/7225; A61B 5/725

(57) ABSTRACT

In view of the foregoing, an aspect herein provides an Electromyography (EMG) sensor (100) further includes electrodes (102) adapted to be attached to an external surface of skin of a limb, and configured to sense signals from the external surface of the skin, a first amplifier (108) connected to the electrodes (102), and configured to generate a first amplified signal based on a differential signal, existing in microvolts, associated with the electrodes (102), a second amplifier (112) connected to the first amplifier (108), and configured to generate a second amplified signal based on the first amplified signal, and a first filter (114) connected to the second amplifier (112), configured to reject high-frequency noise from the second amplified signal to generate an output signal and prevents interference by the electromagnetic interference from nearby electrical noise, false high-frequency signals and in-circuit internal electronic noise. An Electromyography (EMG) system (200) includes the EMG sensor (100) installed in an electronic device, a control circuitry (116) connected to the EMG sensor (100) and configured to control an operation of the electronic device based on the output signal.

### TECHNICAL FIELD

The present invention relates to the field of measurements of biological signals of the body or parts thereof, and more particularly to an electromyography sensor.

### BACKGROUND

Electromyography is a method of measuring the functional state of skeletal muscles based on detection of electric potentials appearing therein. It is used in medicine for diagnosis of peripheral nerves and muscles, including the muscle diseases, for the manufacture of prosthetic limbs and the like. Furthermore, electromyography is used for human-computer interaction (human-computer interaction and muscle-computer interaction), then often electromyographs have portable size and can be used to monitor the status of patients with neuromuscular system, athletes, as well as electronic control units via gestures for human entertainment and everyday life. Painless and portable surface electromyographic signal detection holds significant importance in medical detection methods. At present, the demand for such medical diagnostic and therapeutic instruments on the market is growing rapidly. However, conventional Electromyography (EMG) sensors widely used in prosthetic devices are comparatively substantial in size, made of a rigid structure, and embedded into the cavity to realize contact sensing with the residual limb, which cannot realize flexible adjustment. When the user wears the prosthetic device, the contour and shape of the residual limb in the receiving cavity will change with the wearing action and posture, which will cause the electrode of the electromyographic sensor to separate from the skin, possibly resulting in flawed readings, and affecting the entire prosthetic device based on the EMG signal error and causing overall failure. The EMG sensor also gets affected by the electromagnetic interference (EMI) from nearby electrical noise, false high-frequency signals and in-circuit internal electronic noise. Additionally, the signal received from the electrodes is in microvolts and requires an amplifier.

There is therefore a need in the art for system and method, which overcome above-mentioned and other limitations of existing approaches.

### SUMMARY

In view of the foregoing, an aspect herein provides an Electromyography (EMG) sensor (100) including electrodes (102) adapted to be attached to an external surface of skin of a limb, and configured to sense signals from the external surface of the skin, a first amplifier (108) connected to the electrodes (102), and configured to generate a first amplified signal based on a differential signal associated with the electrodes (102), a second amplifier (112) connected to the first amplifier (108), and configured to generate a second amplified signal based on the first amplified signal, and a first filter (114) connected to the second amplifier (112), and configured to reject high-frequency noise from the second amplified signal to generate an output signal. An Electromyography (EMG) system (200) includes the EMG sensor (100) installed in an electronic device, a control circuitry (116) connected to the EMG sensor (100) and configured to control an operation of the electronic device based on the output signal.

### BRIEF DESCRIPTION OF THE DRAWINGS

Other objects, features, and advantages of the aspect will be apparent from the following description when read with reference to the accompanying drawings. In the drawings, wherein like reference numerals denote corresponding parts throughout the several views:

The diagrams are for illustration only, which thus is not a limitation of the present disclosure, and wherein:

FIG. 1 illustrates a block diagram that depicts an electromyography (EMG) sensor, in accordance with an aspect of the present disclosure;

FIG. 2 illustrates a block diagram of an electromyography (EMG) system including the electromyography sensor of FIG. 1, in accordance with an aspect of the present disclosure; and

FIG. 3 illustrates an internal circuit of the EMG sensor of FIG. 1, in accordance with an aspect of the present disclosure.

To facilitate understanding, like reference numerals have been used, where possible to designate like elements common to the figures.

### DETAILED DESCRIPTION OF THE PREFERRED ASPECTS

The aspects herein and the various features and advantageous details thereof are explained more fully with reference to the non-limiting aspects that are illustrated in the accompanying drawings and detailed in the following description. Descriptions of well-known components and processing techniques are omitted so as to not unnecessarily obscure the aspects herein. The examples used herein are intended merely to facilitate an understanding of ways in which the aspects herein may be practiced and to further enable those of skill in the art to practice the aspects herein. Accordingly, the examples should not be construed as limiting the scope of the aspects herein.

The term “signal”, “sensed signal”, “information signal” and “electromyographic signal” and other such terms indicate the bio-electric signals captured by the EMG sensors (100) when muscles contract and are used interchangeably.

The term “limb” indicates the appendage or a residual appendage of an individual from which the bio-electric signals are being captured via the EMG sensor (100).

The term “differential signal” and other such terms indicate the signal produced by the difference of two input signals.

The term “high frequency noise” indicates the error or undesired random disturbance of a useful information signal and has a frequency of 1000 Hertz (Hz) and above.

The term “operation” indicates the modulation of electronic machines e.g. motors, which convert electric energy into mechanical energy.

The terms “electronic device” or “electronic control units” and other such terms, indicate the instruments utilizing the electromyographic signal e.g. prothesis.

The term “gain” indicates the measure of the ability of an amplifier to increase the power or amplitude of a signal from the input to the output port by adding energy converted from some power supply to the signal.

The term “Impedance” indicates the measure of the total opposition that a circuit or a part of a circuit presents to electric current. It includes both includes both resistance and reactance.

The term “clinical analysis” indicates the scrutiny of the electromyographic signals for the purpose of diagnostic and scientific utilization.

As mentioned, there is a need for the development of a flexible surface EMG sensor capable of amplifying signal from electrodes and filtering noise. The aspect herein overcome the limitations of the prior art by providing an Electromyography (EMG) sensor (100) including electrodes (102) adapted to be attached to an external surface of skin of a limb, and configured to sense signals from the external surface of the skin, a first amplifier (108) connected to the electrodes (102), and configured to generate a first amplified signal based on a differential signal associated with the electrodes (102), a second amplifier (112) connected to the first amplifier (108), and configured to generate a second amplified signal based on the first amplified signal, and a first filter (114) connected to the second amplifier (112), and configured to reject high-frequency noise from the second amplified signal to generate an output signal.

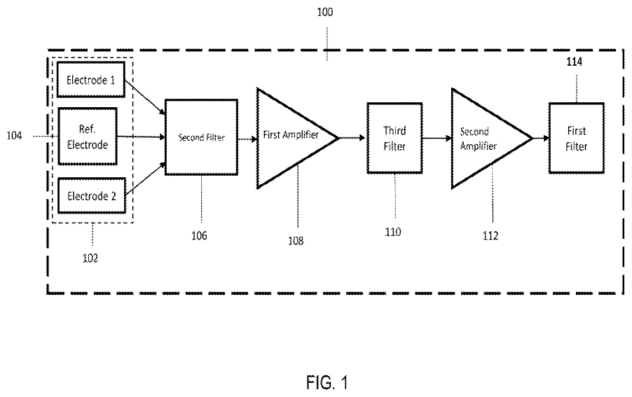

FIG. 1 illustrates the block diagram that depicts the electromyography (EMG) sensor. The EMG sensor (100) includes electrodes (102), a second filter (106), a first amplifier (108), a third filter (110), a post amplifier (112) and a first filter (114).

The electrodes (102) are coupled to the second filter (106), which is coupled to the first amplifier (108), which is in turn coupled to the third filter (110), which is coupled to the second amplifier (112) and the second amplifier is coupled to the first filter (114).

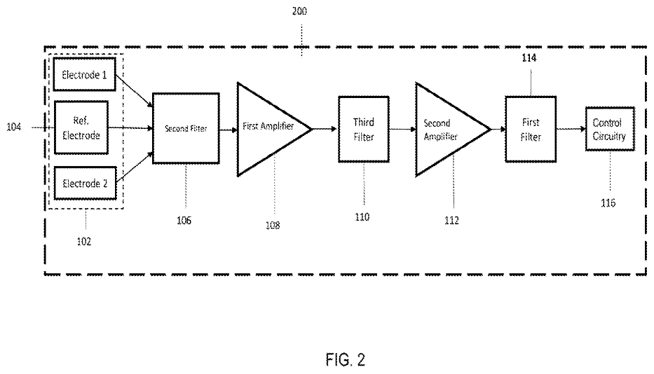

FIG. 2 illustrates a block diagram of an electromyography (EMG) system. Electromyography (EMG) system (200) includes the EMG sensor (100) installed in an electronic device, a control circuitry (116) connected to the EMG sensor (100) and configured to control an operation of the electronic device based on the output signal. The control circuitry (116) is coupled to the first filter (114).

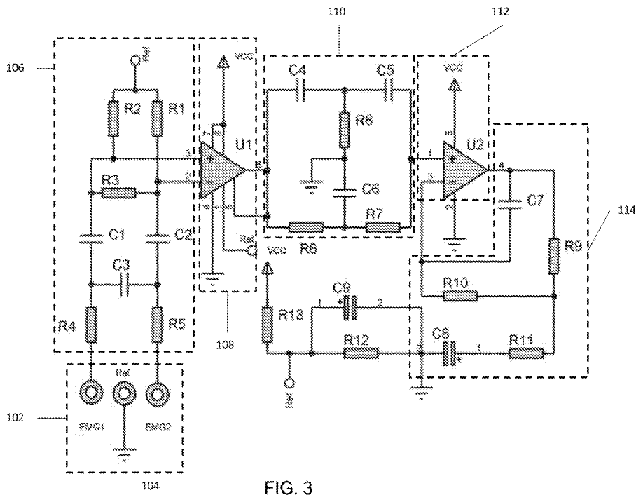

FIG. 3 illustrates an internal circuit of the EMG sensor.

In an aspect, the electrodes (102) are silver plated copper electrodes and an aluminium electrode. In an aspect, the electrodes (102) include two EMG electrodes (EMG1, EMG2) and a reference electrode (104). In another aspect, the sensor may include additional number of electrodes. Each electrode captures the stimulus form the skin of the limb and converts it into electric signals. These signals are then transferred to the input of the second filter (106).

In an aspect, the second filter (104) contains of capacitors C1, C2 of 150 nano-Farad (nF) each and a capacitor C3 of 220 pico-Farad (pF). In an aspect, the second filter (106) further contains of a reference voltage Ref, resistors R1, R2 of 1 Mega-ohm each, a resistor R3 of 220 kilo-ohm and resistors R4, R5 of 2 kilo-ohm each. R4 and R5 are coupled to EMG1 and EMG2 respectively. R1, R2 and C1, C2 and C3 are connected in series, in that order, respectively. R3 exists in parallel with C1, C2 and C3. The second filter (104) rejects the high-frequency electrical signal or noise common to both electrodes at any time instant, while passing the resultant filtered signals to the inputs of the first amplifier (108).

In an aspect, the first amplifier (108) is an 8-pin integrated circuit (IC) and differential amplifier U1. In an aspect, the instrumentation amplifier is coupled to two outputs emerging form the second filter (106) at its input, a reference voltage (Ref), a ground connection, a voltage common collector (VCC) and an output coupled to the input of the third filter (110). The difference between the two inputs is amplified to produce the amplified differential signal at the output and transfer it to the input of the third filter. The VCC is the higher voltage with respect to ground and acts as the power input for the first amplifier (106). In an aspect, the first amplifier (108) possesses very large input impedance in a range of about 1000 Mega-ohms or higher, for the prevention of further decay of raw EMG signal due to the input impedance of the amplifier and is successfully able to amplify the signal, which is in microvolts, received from the electrodes (102). In an aspect, the first amplifier (106) further possesses a fixed gain in a range of about 100-1000 to strengthen the signal for further filtering of desirable signal from the electric signal possessing noise captured by the electrodes (102).

In an aspect, the third filter (110) contains of capacitors C4, C5 of 47 nF each, capacitor C6 of 200 nF and resistors R6, R7 of 68 kilo-ohm, resistor R8 of 33 kilo-ohm and a ground existing between C6 and R8. C4, C6 and R6, R8 are connected in parallel to each other, wherein R8 and C6 are connected in series. C5 and R7 exist at the terminal connections before and after the aforementioned parallel arrangement. The third filter (110) discards any signals not representative of the frequency range of the EMG, which has a frequency range of 0 Hz to about 500 Hz, from the amplified signal at the input and transfers the filtered signal to the primary input of the second amplifier (112).

In an aspect, the second amplifier (112) contains of a 5-pin operational amplifier U2 at the output of the third filter (110). The second amplifier (110) is coupled to the output emerging from the third filter (110) at its primary input at the positive terminal and an additional coupling as feedback loop at its secondary input at the negative terminal, a ground connection, a VCC and an output coupled to the input of the first filter (114). The VCC is the higher voltage with respect to ground and acts as the power input for the second amplifier (112). The difference between the two inputs is amplified to produce the amplified differential signal at the output coupled to the input of the first filter (114).

In an aspect, the first filter (114) contains of capacitor C7 of 2 nF, a Tantalum Capacitor C8 of 4.7 micro-Farad (μF), a resistor R9 of 68 kilo-ohm, a resistor R11 of 2 kilo-ohm and a feedback resistor R10 of 2 kilo-ohm. C7 exists in parallel with R9 and R10. R11 and C8 are connected in series and coupled to the aforementioned parallel arrangement via R11. The first filter (114) levels the output of the second amplifier and discards all high-frequency noise from the output which is connected to the input of the control circuitry (116). The implementation of the 3 filters included in the EMG sensor (100) prevents interference by the EMI from nearby electrical noise, false high-frequency signals and in-circuit internal electronic noise.

In an aspect, the EMG sensor (100) further contains of a ground connection, a VCC, resistor R12 of 18 kilo-ohm, resistor R13 of 24 kilo-ohm, a Ref and a tantalum capacitor C9 of 10 μF. R13 is connected in series with the parallel arrangement of C9, R12 and coupled to the VCC and Ref at each terminal connection respectively. The resistors R12 and R13 act as the reference voltage and the capacitor C9 stabilizes these reference voltages.

In an aspect, the components of the EMG sensor (100) are housed in a flexible PCB module. The flexible nature of the housing prevents the separation of the EMG sensor (100) from the skin with change in the contour and shape of the residual limb and enables the EMG sensor (100) to realize flexible adjustment. In an aspect, the flexible PCB module has a dimension of 26×9 millimetre (mm), a thickness of 0.05 mm to 0.8 mm and 4 layers. Additionally, said module does not require a model case. Such a housing enables the EMG sensor (100) to occupy less space than conventional sensors. In another aspect, the surface EMG sensor can also be housed in a rigid PCB module with equal dimension, a thickness of 0.3 mm to 1.6 mm, 2 layers and requires a model case.

In an aspect, the control circuitry (116) processes the signal transferred by the EMG sensor (100). The control circuitry (116) is responsible interpreting data from the signal for further utilization in clinical analysis or electronic control unit. It remains in active calibration at regular intervals to dynamically adjust the gain and threshold from the EMG sensor (100).

We claim:

1. An Electromyography (EMG) sensor (100) containing:
electrodes (102) adapted to be attached to an external surface of skin of a limb, and configured to sense signals from the external surface of the skin;
a first amplifier (108) coupled to the electrodes (102), and configured to generate a first amplified signal based on a differential signal associated with the electrodes (102);
a second amplifier (112) coupled to the first amplifier (108), and configured to generate a second amplified signal based on the first amplified signal; and
a first filter (114) coupled to the second amplifier (112) and configured to reject high-frequency noise from the second amplified signal to generate an output signal, wherein the output signal facilitates to control an operation of an electronic device.

2. The EMG sensor (100) as claimed in claim 1, further containing a second filter (106) coupled to each electrode of the electrodes (102) and the first amplifier (108), and configured to provide the differential signal associated with the electrodes (102) based on the sensed signals.

3. The EMG sensor (100) as claimed in claim 2, wherein the second filter (106) is further configured to reject at a specific time instant, a common signal associated with the electrodes (102) based on the sensed signals.

4. The EMG sensor (100) as claimed in claim 1, further containing a third filter (110) coupled to the first amplifier (108) and the second amplifier (112), and configured to provide the first amplified signal to the second amplifier (112) such that the first amplified signal has a predefined frequency.

5. The EMG sensor (100) as claimed in claim 4, wherein the predefined frequency is in a range from about 1 Hertz (Hz) to about 500 Hz.

6. The EMG sensor (100) as claimed in claim 1, further containing a reference electrode (104) adapted to be attached to a reference point at the external surface of the skin and configured to provide a reference signal such that the sensed signals are measured with respect to the reference signal.

7. The EMG sensor (100) as claimed in claim 1, wherein the first amplifier (108) has (i) a gain in a range of about 100-1000 and (ii) a large input impedance in a range of 100-1000 Mega-ohms or higher.

8. The EMG sensor (100) as claimed in claim 1, wherein the first filter (114) is further configured to provide the output signal to control circuitry (116) such that the control circuitry (116) controls the operation of the electronic device based on the output signal.

9. The EMG sensor (100) as claimed in claim 8, wherein to control the operation of the electronic device, the control circuitry (116) to process the output of the EMG system for clinical analysis and electronic control units.

10. The EMG sensor (100) as claimed in claim 1, wherein each electrode of the electrodes (102) is selected from one of, a silver-plated copper electrode and an aluminum electrode.

11. An Electromyography (EMG) system (200) containing:
an Electromyography (EMG) sensor (100) installed in an electronic device, the EMG sensor (100) containing:
electrodes (102) adapted to be attached to an external surface of skin of a limb, and configured to sense signals from the external surface of the skin;
a first amplifier (108) coupled to the electrodes (102), and configured to generate a first amplified signal based on a differential signal associated with the electrodes (102);
a second amplifier (112) coupled to the first amplifier (108), and configured to generate a second amplified signal based on the first amplified signal; and
a first filter (114) coupled to the second amplifier (112), and configured to reject high-frequency noise from the second amplified signal to generate an output signal; and
control circuitry (116) coupled to the EMG sensor (100) and configured to control an operation of the electronic device based on the output signal.

12. The EMG system (200) as claimed in claim 11, further containing a second filter (106) coupled to each electrode of the electrodes (102) and the first amplifier (108), and configured to provide, to the first amplifier (108), the differential signal associated with the electrodes (102) based on the sensed signals.

13. The EMG system (200) as claimed in claim 11, wherein the noise cancellation circuit (108) is further configured to reject, at a specific time instant, a common signal associated with the electrodes (102) based on the sensed signals.

14. The EMG system (200) as claimed in claim 11, further containing a third filter (110) coupled to the first amplifier (108) and the second amplifier (112), and configured to provide the first amplified signal to the second amplifier (112) such that the first amplified signal has a predefined frequency.

15. The EMG system (200) as claimed in claim 14, wherein the predefined frequency is in a range from about 1 Hertz (Hz) to about 500 Hz.

16. The EMG system (200) as claimed in claim 11, further containing a reference electrode (104) adapted to be attached to a reference point of the external surface of the skin, and configured to provide a reference signal such that the sensed signals are measured with respect to the reference signal.

17. The EMG system (200) as claimed in claim 11, wherein the first amplifier (108) has (i) a gain in a range of about 100 to 1000 and (ii) a large input impedance in range of about 1000 Mega-ohms or higher.

18. The EMG system (200) as claimed in claim 11, wherein to control the operation of the electronic device, the control circuitry (116) is configured to process the output of the EMG system for clinical analysis and electronic control units.

19. The EMG system (200) as claimed in claim 11, wherein each electrode of the electrodes (102) is selected from one of, a silver-plated copper electrode and an aluminum electrode.
